(19) United States
(12) Patent Application Publication
(10) Pub. No.: US 2006/0111232 A1
(43) Pub. Date: May 25, 2006

(54) Multi-staged wax displacement process for catalyst recovery from a slurry

(75) Inventors: Michael D. Spena, Katy, TX (US); Doug S. Jack, Ponca City, OK (US); Dan Fraenkel, Boulder, CO (US)

Correspondence Address: DAVID W. WESTPHAL;CONOCOPHILLIPS COMPANY - I.P. Legal, P.O. BOX 1267, PONONCA CITY, OK, 74602-1267, US

(73) Assignee: ConocoPhillips Company, Houston, TX (US)

(21) Appl. No.: 10/994,428

(22) Filed: Nov. 22, 2004

(51) Int. Cl. B01J 20/34; C07C 1/04

(52) U.S. Cl. 502/29; 518/709

(57) ABSTRACT

In a system and method for cleaning and recovering a solid catalyst, a solvent is added to a slurry comprising the catalyst and residual hydrocarbons. A portion of the residual hydrocarbons and the solvent are separated from the slurry, and the solvent addition and separating steps are repeated until a desired amount of the hydrocarbons have been removed. The solvent is finally removed from the catalyst using a stripping gas to recover a cleaned catalyst. In preferred embodiments, the slurry comprises a Fischer-Tropsch solid catalyst; the slurry exits a slurry bubble reactor, or both. In an embodiment, the solvent comprises naphtha; the residual hydrocarbons comprise waxy hydrocarbons; and one of the separation steps comprises filtration. In an embodiment, the repeating of the solvent addition and separating steps is carried out in a plurality of filters operated in series.

### CROSS-REFERENCE TO RELATED APPLICATIONS

This application is related to U.S. patent application Ser. No. ______ (attorney docket number 1856-33900 (9742.0-02)) filed concurrently herewith and entitled “A Process for Catalyst Recovery from a Slurry Containing Residual Hydrocarbons,” which is incorporated by reference herein in its entirety.

### FIELD OF THE INVENTION

This invention generally relates to the recovery of a solid catalyst from a hydrocarbon liquid, and cleaning of said solid catalyst, particularly by removal of wax residue from Fischer-Tropsch catalyst particles dispersed in a slurry. More specifically, the invention relates to a process for removing residual hydrocarbons from a metal catalyst by adding a solvent to a slurry of hydrocarbons and metal catalyst particles, separating the hydrocarbons and solvent from the catalyst, and recovering the catalyst for reclamation.

### BACKGROUND OF THE INVENTION

Natural gas, found in deposits in the earth, is an abundant energy resource. For example, natural gas commonly serves as a fuel for heating, cooking, and power generation, among other things. The process of obtaining natural gas from an earth formation typically includes drilling a well into the formation. Wells that provide natural gas are often remote from locations with a demand for the consumption of the natural gas.

Thus, natural gas is conventionally transported large distances from the wellhead to commercial destinations in pipelines. This transportation presents technological challenges due in part to the large volume occupied by a gas. Because the volume of a gas is so much greater than the volume of a liquid containing the same number of gas molecules, the process of transporting natural gas typically includes chilling and/or pressurizing the natural gas in order to liquefy it. However, this contributes to the final cost of the natural gas.

Further, naturally occurring sources of crude oil used for liquid fuels such as gasoline and middle distillates have been decreasing and supplies are not expected to meet demand in the coming years. Middle distillates typically include heating oil, jet fuel, diesel fuel, and kerosene. Fuels that are liquid under standard atmospheric conditions have the advantage that in addition to their value, they can be transported more easily in a pipeline than natural gas, since they do not require energy, equipment, and expense required for liquefaction.

Thus, for all of the above-described reasons, there has been interest in developing technologies for converting natural gas to more readily transportable liquid fuels, i.e. to fuels that are liquid at standard temperatures and pressures. One method for converting natural gas to liquid fuels involves two sequential chemical transformations. In the first transformation, natural gas or methane, the major chemical component of natural gas, is reacted with oxygen to form syngas, which is a combination of carbon monoxide gas and hydrogen gas. In the second transformation, known as the Fischer-Tropsch process, carbon monoxide is reacted with hydrogen to form organic molecules containing carbon and hydrogen. Those organic molecules containing only carbon and hydrogen are known as hydrocarbons. In addition, other organic molecules containing oxygen in addition to carbon and hydrogen known as oxygenates may be formed during the Fischer-Tropsch process. Hydrocarbons having carbons linked in a straight chain are known as aliphatic hydrocarbons that may include paraffins and/or olefins. Paraffins are particularly desirable as the basis of synthetic diesel fuel.

Typically the Fischer-Tropsch product stream contains hydrocarbons having a range of numbers of carbon atoms, and thus having a range of molecular weights. Thus, the Fischer-Tropsch products produced by conversion of natural gas commonly contain a range of hydrocarbons including gases, liquids and waxes. Depending on the molecular weight product distribution, different Fischer-Tropsch product mixtures are ideally suited to different uses. For example, Fischer-Tropsch product mixtures containing liquids may be processed to yield gasoline, as well as heavier middle distillates. Hydrocarbon waxes may be subjected to an additional processing step for conversion to liquid and/or gaseous hydrocarbons. Thus, in the production of a Fischer-Tropsch product stream for processing to a fuel it is desirable to maximize the production of high value liquid hydrocarbons, such as hydrocarbons with at least 5 carbon atoms per hydrocarbon molecule (C₅₊ hydrocarbons).

The Fischer-Tropsch process is commonly facilitated by a catalyst. Catalysts desirably have the function of increasing the rate of a reaction without being consumed by the reaction. A feed containing carbon monoxide and hydrogen is typically contacted with a catalyst in a reaction zone that may include one or more reactors.

Common reactors include packed bed (also termed fixed bed) reactors, fluidized bed reactors and slurry bed reactors. Originally, the Fischer-Tropsch synthesis was carried out in packed bed reactors. These reactors have several drawbacks, such as temperature control, that can be overcome by gas-agitated slurry reactors or slurry bubble column reactors. Gas-agitated multiphase reactors sometimes called “slurry reactors” or “slurry bubble columns,” operate by suspending catalytic particles in liquid and feeding gas reactants into the bottom of the reactor through a gas distributor, which produces gas bubbles. As the gas bubbles rise through the reactor, the reactants are absorbed into the liquid and diffuse to the catalyst where, depending on the catalyst system, they are typically converted to gaseous and liquid products. The gaseous products formed enter the gas bubbles and are collected at the top of the reactor. Liquid products are recovered from the suspending liquid by using different techniques like filtration, settling, hydrocyclones, magnetic techniques, etc. Gas-agitated multiphase reactors or slurry bubble column reactors (SBCRs) inherently have very high heat transfer rates, and therefore, reduced reactor cost. This, and the ability to remove and add catalyst online are some of the principal advantages of such reactors as applied to the exothermic Fischer-Tropsch synthesis. Sie and Krishna (Applied Catalysis A: General 1999, 186, p. 55), incorporated herein by reference in its entirety, give a history of the development of various Fischer Tropsch reactors.

Typically, in the Fischer-Tropsch synthesis, the distribution of weights that is observed such as for C₅₊ hydrocarbons, can be described by likening the Fischer-Tropsch reaction to a polymerization reaction with an Anderson-Shultz-Flory chain growth probability (α) that is independent of the number of carbon atoms in the lengthening molecule. α is typically interpreted as the ratio of the mole fraction of Cₙ₊₁ product to the mole fraction of Cₙ product. A value of a of at least 0.72 is preferred for producing high carbon-length hydrocarbons, such as those of diesel fractions.

The composition of a catalyst influences the relative amounts of hydrocarbons obtained from a Fischer-Tropsch catalytic process. Common catalysts for use in the Fischer-Tropsch process contain at least one metal from Groups 8, 9, or 10 of the Periodic Table (in the new IUPAC notation, which is used throughout the present specification).

Cobalt metal is particularly desirable in catalysts used in converting natural gas to heavy hydrocarbons suitable for the production of diesel fuel. Alternatively, iron, nickel, and ruthenium have been used in Fischer-Tropsch catalysts. Nickel catalysts favor termination and are useful for aiding the selective production of methane from syngas. Iron has the advantage of being readily available and relatively inexpensive but the disadvantage of a high water-gas shift activity by which carbon monoxide, instead of reacting with hydrogen to produce hydrocarbons, is rejected from the system as the undesired carbon dioxide while forming, rather than consuming, hydrogen (from the water). Ruthenium has the advantage of high activity but is quite expensive and scarce.

Many petroleum and chemical processes use particulate catalysts for the conversion of a feedstock to one or more desired products, such as the Fischer-Tropsch process described herein. In any reaction requiring a catalyst, the catalyst can be expected to have a certain life, for example several months to a few years. Accordingly, as the time on stream increases, the catalyst tends to degrade and eventually becomes ineffective. The spent catalyst or a portion of the spent catalyst can be removed from a reactor vessel, and in order to maintain catalyst inventory into the reactor, new and/or regenerated catalyst can be loaded therein. The selection of a catalyst depends largely on the cost of manufacture of the catalyst and the ability for the catalyst activity to be restored. The removed spent catalyst can undergo a regeneration process if the activity of the spent catalyst removed from the reactor vessel can be at least partially restored. However, in some cases the loss of catalyst activity is irreversible and the spent catalyst can undergo a reclamation process to recover the valuable materials. While such reclamation processes may be located on site, they are often off site and the spent catalyst must be transported for processing. In such cases, it is preferable to remove any residual hydrocarbonaceous products from the spent catalyst prior to processing the spent catalyst in order to recover the value of the hydrocarbonaceous products, avoid the additional transportation costs associated with the weight of the residual hydrocarbonaceous products, and minimize the presence of hydrocarbonaceous compounds in any waste materials for environmental conservation reasons. Therefore, a need exists in the art for efficient methods and systems for the removal of residual hydrocarbonaceous products from catalysts, and in particular Fischer-Tropsch catalysts that are used in large quantities, to facilitate the reclamation of such catalysts.

### SUMMARY OF THE INVENTION

A method for cleaning and recovering a solid catalyst is disclosed, said method comprising the following steps: (a) providing a plurality of solvent streams and a plurality of solid-liquid separation units operated in series; (b) forming a slurry feedstream by adding one of the plurality of solvent streams to a slurry stream comprising a solid catalyst and residual hydrocarbons; (c) passing the slurry feedstream through a solid-liquid separation unit in the series so as to generate a retentate slurry stream and a liquid stream, wherein the retentate slurry stream comprises the solid catalyst, a portion of the solvent and a lesser content in residual hydrocarbons than that in the slurry feedstream, and wherein the liquid stream comprises a portion of the residual hydrocarbons and the other portion of the solvent; (d) repeating steps (b) and (c) for each of the downstream solid-liquid separation units in the series, wherein the slurry feedstream for each of the downstream solid-liquid separation units is formed by adding one of the plurality of solvent streams to the retentate slurry stream exiting the liquid-solid separation unit located immediately upstream of said downstream solid-liquid separation unit, said repeating being performed until a desired amount of the residual hydrocarbons is removed from the slurry stream and a catalyst stream exiting the last solid-liquid separation unit in the series comprises primarily the solid catalyst and some solvent; (e) removing the solvent from the catalyst stream using a stripping gas under a suitable temperature so as to vaporize the solvent and form a cleaned solid catalyst; and (f) collecting the cleaned solid catalyst. In an embodiment, the catalyst is a Fischer-Tropsch catalyst contained in a reactor, for example preferably a slurry bubble reactor or alternatively a fixed bed reactor. In some embodiments, the solvent is naphtha. In some embodiments, the hydrocarbons comprise waxy hydrocarbons. In other embodiments, the solvent is naphtha, and the hydrocarbons comprise waxy hydrocarbons. In an embodiment, the separating step is performed by filtering, for example cross flow filtration, rotary filtration, cake filtration or both. In an embodiment, the repeating of the solvent addition and separating steps is carried out in a plurality of filters in series. In an embodiment, more than 90 percent of the hydrocarbons are removed from the slurry feedstream. In another embodiment, more than 95 percent of the hydrocarbons are removed from the slurry feedstream. In yet other embodiment, substantially all of the hydrocarbons are removed from the slurry feedstream.

A system for cleaning a solid catalyst comprising wax residue to obtain a cleaned catalyst suitable for reclamation, is disclosed, said system comprising: means for diluting a slurry comprising a solid catalyst and residual hydrocarbons with a solvent; a plurality of solid-liquid separation units in series configured for receiving a diluted slurry and separating substantially all of the residual hydrocarbons from the solid catalyst so that each of the plurality of solid-liquid separation units forms a liquid stream and a retentate slurry, wherein the diluted slurry for any of the downstream solid-liquid separation units comprises the retentate slurry from the solid-liquid separation unit immediately upstream of said downstream solid-liquid separation unit; and a stripper connected to the last solid-liquid separation unit in the series for receiving the retentate slurry exiting from the last solid-liquid separation unit and configured for passing a stripping gas so as to remove any residual hydrocarbons and solvent from the solid catalyst present in the retentate slurry exiting from the last solid-liquid separation unit and form a cleaned catalyst. In an embodiment, the slurry is provided preferably from a slurry bubble Fischer-Tropsch reactor or alternatively from a fixed bed Fischer-Tropsch reactor, or a manifolded plurality of such Fischer-Tropsch reactors. In an embodiment, at least one of the solid-liquid separation units employs filtration; In preferred embodiments, the plurality of solid-liquid separation units employs filtration; more preferably, the solid-liquid separation units comprises filters, such as rotary filters, cross flow filters, cake filters, and any combination thereof. In an embodiment, the system further comprises a degasser connected to the stripper and configured for receiving and degassing any vaporized hydrocarbons, solvent, and/or stripping gas remaining with the catalyst.

An integrated process for producing hydrocarbons is disclosed, said integrated process comprising the following steps: (a) contacting a synthesis solid catalyst with a feed stream comprising carbon monoxide and hydrogen in a reaction zone within a reactor to produce hydrocarbon products until all or a portion of the solid catalyst needs to be replaced; (b)removing all or a portion of the solid catalyst from the reactor via a slurry comprising said solid catalyst and hydrocarbons; (c) adding a solvent to the slurry to form a diluted slurry; (d) separating a portion of the hydrocarbons and the solvent from the diluted slurry; (e) repeating steps (c) and (d) until a desired amount of the hydrocarbons have been removed such as to generate a catalyst stream comprising primarily the solid catalyst and some solvent; (f) removing the solvent from the catalyst stream using a stripping gas to form a cleaned solid catalyst; (g) recovering the cleaned solid catalyst; and (h) replacing the removed catalyst with fresh catalyst. In preferred embodiments, the slurry is provided from one slurry bubble Fischer-Tropsch reactor, or a manifolded plurality of such Fischer-Tropsch reactors.

The invention further relates to a method for unloading the content of a slurry bubble reactor comprising a solid catalyst and a waxy hydrocarbon liquid with minimal solidification of the waxy hydrocarbon liquid to recover the solid catalyst, the method comprising the steps of (a) passing a feed gas comprising hydrogen and carbon monoxide as reactant gases through a slurry being maintained in the reactor under conversion promoting conditions which include a reaction temperature between about 160° C. and 300° C. to convert at least a portion of said reactant gases to hydrocarbon products, wherein the slurry comprises a solid catalyst and a molten waxy hydrocarbon liquid; (b) substituting one or both of the reactant gases by an unreactive gas or removing one of the reactant gases so as to stop the hydrocarbon synthesis reaction; (c) adding a lighter diluting hydrocarbon liquid to the slurry in the reactor so as to gradually reduce the content of the molten waxy hydrocarbon liquid in the slurry; (d) optionally, cooling the slurry within the slurry bubble column reactor from the reaction temperature to a lower temperature; (e) withdrawing a slurry stream from the reactor and passing the slurry stream through an external slurry circulation loop comprising a solid-liquid separation unit to form a solid-enriched slurry stream and a hydrocarbon product stream which exits the external slurry circulation loop; (f) recycling the majority of or all of the solid-enriched slurry stream to said slurry bubble column reactor; (g) performing steps (c), (e), (f) and optionally (d) until the slurry contained in the reactor has a lower waxy hydrocarbon content and has an acceptable temperature without causing solidification of the slurry within the reactor; and (h) withdrawing a part of or all of the wax-reduced slurry from the reactor to feed a recovery system to recover the solid catalyst therein. In some embodiments, the acceptable temperature ranges from ambient temperature to about 160° C. In other embodiments, the acceptable temperature ranges from ambient temperature to about 120° C. In some embodiments, step (c) is performed continuously or intermittently. In some embodiments, step (d) is performed continuously or intermittently. In other embodiments, step (d) is performed while step (c) is performed. In additional or alternate embodiments, step (d) is performed before step (b). In other embodiments, the lighter diluting hydrocarbon liquid comprises a hydrocarbon mixture within the naphtha, diesel, or kerosene boiling range. In yet other embodiments, the lighter diluting hydrocarbon liquid comprises a hydrocarbon mixture within the diesel boiling range. In some embodiments, the solid-liquid separation unit comprises a filter. In some embodiments, the external slurry circulation loop further comprises a degasser to remove entrapped gas from the slurry stream. In other embodiments, the external slurry circulation loop comprises a degasser and a filter, wherein the degasser forms a degassed slurry stream, and the filter is adapted to receive the degassed slurry stream.

### BRIEF DESCRIPTION OF THE DRAWINGS

The invention, together with further advantages thereof, may best be understood by reference to the following description taken in conjunction with the accompanying drawings in which:

FIG. 1 represents a process flow diagram of an embodiment of a catalyst recovery process according to the present invention comprising a plurality of solid/liquid separation units; means for providing a solvent to the plurality of solid/liquid separation units; and an optional stripping zone, wherein the separation of a solid catalyst from a catalyst slurry stream comprising waxy hydrocarbons is facilitated by the displacement of waxy hydrocarbons by the solvent;

FIG. 2 illustrates embodiments of different means of providing a catalyst slurry stream to the catalyst recovery process according to the present invention, said means comprising a catalytic reaction system and/or a solid-liquid separation unit connected to said catalytic reaction system;

FIG. 3 illustrates one alternate embodiment for a means of providing a catalyst slurry stream for the catalyst recovery process according to the present invention, said means comprising a liquid-liquid extraction unit and a solid-liquid separation unit; and

FIG. 4 illustrates yet another alternate embodiment for a means of providing a catalyst slurry stream to the catalyst recovery process according to the present invention, said means comprising a solid-liquid separation unit.

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present invention is susceptible to embodiments of different forms. There are shown in the drawings, and herein will be described in detail, specific embodiments of the present invention with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results. Specifically, the catalyst recovery process and system of the present invention may be used with any suitable catalyzed synthesis reaction wherein the catalyst needs to be cleaned of residual hydrocarbons prior to subsequent processing. In an embodiment, the catalyst recovery process and system of the present invention is integrated with a synthesis reaction for producing hydrocarbon liquids from hydrocarbon gas, for example a Fischer-Tropsch synthesis reaction or an alcohol (e.g., methanol) synthesis reaction. In another embodiment, the catalyst recovery process and system of the present invention is integrated with a Fischer-Tropsch synthesis reaction for converting syngas to hydrocarbon liquids via contact with a Fischer-Tropsch catalyst, and the remainder of the detailed description will focus on this embodiment with the understanding that the present invention may have broader applications.

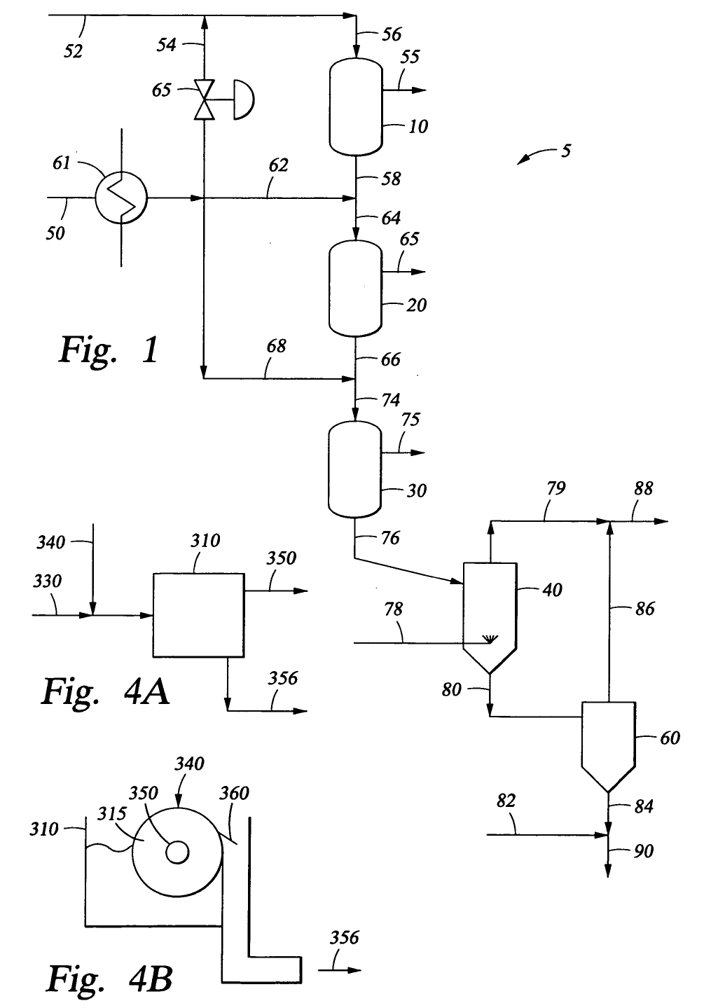

In an embodiment shown in FIG. 1, the catalyst recovery system 5 for recovering a solid catalyst is comprised of a plurality of solid/liquid separation units 10, 20, and 30 in series and an optional stripping vessel 40. Any suitable solid/liquid separation unit, means, or combinations thereof may be employed as solid/liquid separation units 10, 20, and 30. Solid-liquid separation techniques may be used such as filtration, decantation, sedimentation, centrifugation, magnetic separation, or any combination thereof. For example, each of the solid/liquid separation units 10, 20, and 30 could comprise a settler, a hydrocyclone, magnetic separation unit, a centrifuge, a filter or a combination of two or more thereof. In preferred embodiments, solid/liquid separation units 10, 20, and 30 comprise a filter. Any suitable filter or filtration means may be used in units 10, 20, and 30 to separate the liquid and solid catalyst. The filter may be selected from the group consisting of rotary filter, cross-flow filter, cake filter, and any combination of two or more thereof. In some embodiments, cake filtration occurs according to the methods and apparatus in U.S. Patent Application Publication 2003/0232894, entitled “Optimized Solid/Liquid Separation System for Multiphase Converters” and in WIPO published patent application WO 03/089,102, entitled “Solid/liquid Separation System for Multiphase Converters”, each of which is incorporated by reference herein in its entirety.

In an embodiment wherein separation units 10, 20, and 30 are filters, a Fischer-Tropsch slurry is drawn across a filter medium composed of a filter cake disposed on a filter substrate, such that the filter cake forms a primary filter. The thickness of the filter cake is maintained within a desired range by controlling the velocity of the slurry flowing across the cake. The velocity of the slurry across filter cake is preferably maintained between 0.1 feet/second and 4.0 feet/second in order to maintain a preferred filter cake. An instantaneous velocity of the slurry exceeding 5.0 feet/second may result in a loss of thickness of filter cake. The filter medium is resistant to the deleterious effects of ultra-fine catalyst particles bypassing the filter medium and being lost in the liquid products.

The catalyst recovery system 5 for recovering a solid catalyst preferably includes a means for diluting a slurry feedstream before it is fed to one of the solid/liquid separation units 10, 20, and 30, wherein the slurry feedstream to each of the solid/liquid separation units comprises a solid catalyst disposed in a hydrocarbon liquid. The catalyst recovery system 5 is generally fed by a solvent feed 50 and by a slurry stream 52. Solid/liquid separation unit 10 has an inlet for receiving a slurry feedstream 56 (which comprises slurry stream 52) and two outlets 55 and 58, one for a liquid stream and the other for the catalyst-containing slurry stream, respectively. The remaining solid/liquid separation units 20 and 30 are configured for receiving a catalyst-containing slurry stream from an upstream separation unit and for feeding its catalyst-containing slurry stream to a downstream separation unit. The optional stripper 40 is preferably connected to the last separation unit 30 and is configured for receiving a catalyst-containing slurry stream exiting unit 30 and for adding stripping gas therein to strip any residual hydrocarbons and solvent from the catalyst. The catalyst recovery system 5 may further comprise a degasser 60 connected to the stripper 40 and configured for receiving and degassing any vaporized hydrocarbons, solvent, or stripping gas remaining with the catalyst.

In the embodiment of FIG. 1, a solvent is added to dilute the waxy hydrocarbons in the slurry stream so as to displace the heavy waxy hydrocarbons with lighter hydrocarbons, thus lowering the boiling point of the hydrocarbon mixture. The solvent may be any hydrocarbon liquid having a boiling point preferably below the temperature of a hot gas stream, e.g., stream 78, used in a later stripping step to boil off the solvent.

Solvent feed 50 should comprise a hydrocarbon liquid stream, which includes at least one hydrocarbonaceous compound having a carbon number greater than about 5 but having a boiling point not greater than about 650° F. (about 345° C.). Preferably, solvent feed 50 comprises a mixture of hydrocarbonaceous compounds. The mixtures of hydrocarbonaceous compounds may be selected from any suitable source such as a fraction or portion of a Fischer-Tropsch liquid product from one or more Fischer-Tropsch reactors; a liquid fraction from a product upgrading unit; or any hydrocarbon stream in the C₅ to C₁₁ range, preferably in the C₅ to C₉ range or the C₆ to C₉ range. Solvent feed 50 preferably comprises naphtha, diesel, natural gas liquids (NGL), or any combination thereof. The use of light hydrocarbons, such as in naphtha boiling range between about 70° F. and about 350° F. (about 20-175° C.) are more preferred components of solvent feed 50. In some embodiments, solvent feed 50 is naphtha, which is supplied from the product refining/upgrading unit. In most preferred embodiments, solvent feed 50 comprises at least a portion of or a fraction of a Fischer-Tropsch liquid product; most particularly a Fischer-Tropsch naphtha. The use of the concepts of the present invention is not limited however to using Fischer-Tropsch liquid products and can be used with any hydrocarbon liquid exhibiting beneficial properties taken from any readily available source, including product storage tanks if necessary. While the solvent feed 50 described in embodiment of FIG. 1 is described herein as comprising naphtha, it should be understood that any suitable solvent or combination of solvents may be used herein, since it is envisioned that solvent feed 50 may also comprise heavier hydrocarbons, such as Fischer-Tropsch diesel hydrocarbons typically with a boiling range between about 350° F. and about 650° F. (about 175-345° C.) and typically comprising between about 10 and 21 carbon numbers, with some minor content of hydrocarbons with higher than 21 carbons and lower than 10 carbons.

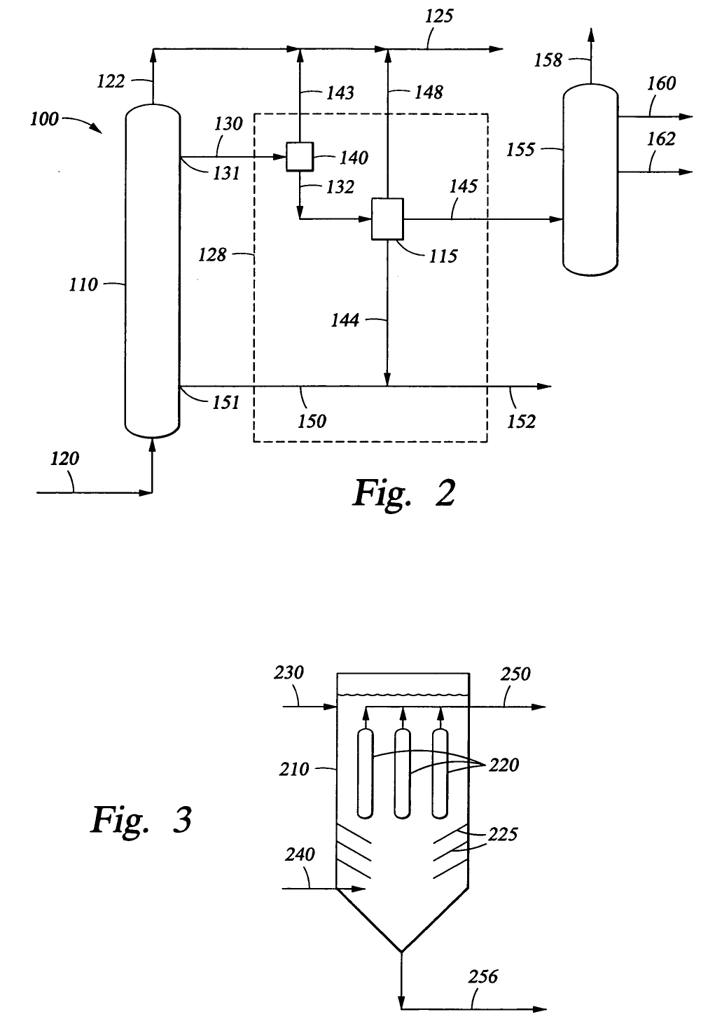

Providing the slurry stream 52 to catalyst recovery system 5 preferably comprises withdrawing a catalyst-containing slurry stream from a reaction vessel, preferably from a slurry bubble column reactor, more preferably from a slurry bubble column Fischer-Tropsch reactor. Providing the slurry stream 52 may comprise altering the composition of a catalyst-containing slurry stream withdrawn from the reaction vessel. Hence, catalyst-containing slurry stream 52 may be provided directly ‘as is’ from a reaction vessel to the unit 10; or alternatively, catalyst-containing slurry stream 52 withdrawn from a reaction vessel may be diluted with a solvent stream 54 to form the slurry feedstream 56 as to change the slurry solid content, the composition of the dispersing hydrocarbon liquid in the slurry, the residual hydrocarbon content, or any combination thereof. Some different possible means for providing the slurry stream 52 to catalyst recovery system 5 are described herein, and some of these means are illustrated in FIGS. 2 and 3.

Slurry stream 52 may be provided continuously from a hydrocarbon synthesis process (such as a process employing the Fischer-Tropsch synthesis), or may be provided in a batch mode or semi-batch mode, for example from one or more storage tanks or holding tanks. Slurry stream 52 may be provided from one hydrocarbon synthesis reactor or a plurality of reactors. Slurry stream 52 preferably comprises finely divided solids such as catalyst particles disposed in a waxy hydrocarbon liquid. The solid content in slurry stream 52 typically comprises from about 10 to about 40% of the total weight of the slurry. Slurry stream 52 typically has a temperature sufficiently high to maintain its waxy hydrocarbon components in a molten state.

Solid/liquid separation unit 10 is configured for receiving slurry feedstream 56. One means for providing slurry feedstream 56 is illustrated in an embodiment of FIG. 1. The slurry feedstream 56 is preferably formed by combining solvent stream 54 (i.e., a portion of solvent feed 50) and slurry stream 52 (preferably derived from a synthesis reactor, such as from a Fischer-Tropsch reactor 110 illustrated in FIG. 2 and described later). The combination of streams 52 and 54 to form slurry feedstream 56 may be performed to adjust the solid catalyst content of the resulting slurry feedstream 56, and/or the slurry flowability (such as reducing the viscosity of the slurry stream) and/or the slurry temperature to be within a more desirable range before the slurry feedstream 56 is fed to separation unit 10. The solvent stream 54 may be also added to slurry stream 52 to improve the transportability (pumpability) of the slurry feedstream 56 to separation unit 10. The solid content in slurry feedstream 56 should be between about 5 percent by weight (wt %) and about 30 wt % of the total weight of the slurry, preferably between about 10 wt % and about 25 wt %. In preferred embodiments, solvent stream 54 is added to slurry stream 52 so as to displace at least some of the waxy hydrocarbons from the solid (catalyst) surface and dissolve the displaced waxy hydrocarbons into the diluted hydrocarbon liquid in slurry feedstream 56. In order to promote the subsequent vaporization of the solvent later in the process in optional stripper 40, solvent stream 54 preferably is added to slurry stream 52 at a temperature of from about 350° F. to about 450° F. (about 175-230° C.). Additionally, the temperature of slurry feedstream 56 preferably is at least 250° F. (about 120° C.) to prevent wax crystallization from occurring during operation of solid-liquid separation unit 10.

If the supplied solvent feed 50 has a temperature which falls within the desired temperature range, no preheating of solvent feed 50 is required. Typically, naphtha is supplied from a refining/upgrading process (not illustrated) at a temperature of from about 190° F. to 200° F. (about 83-93° C.). Therefore, to achieve the preferred temperature of from about 350° F. to 450° F., solvent feed 50 comprising naphtha may be preheated in optional heater 61 (as shown in FIG. 1) for example in a heat exchanger employing steam. The flow rate of solvent feedstream 54 added to the slurry stream 52 can be regulated by valve 65 in order to achieve the desired property or properties in slurry feedstream 56.

Slurry stream 52 typically has a temperature of about 200° F. to about 550° F. (about 90-290° C.), preferably a temperature of about 320° F. to about 550° F. (about 160-290° C.), more preferably a temperature of about 320° F. to about 480° F. (about 160-250° C.). Solvent stream 54 added to dilute slurry stream 52 to form slurry feedstream 56 is preferably characterized by a temperature such as to prevent crystallization of waxy components in slurry feedstream 56. Preferably, the temperature of solvent stream 54 is equal to or higher than that of slurry stream 52 before combining. If solvent stream 54 has a temperature below the desired temperature range, solvent stream 54 may be preheated, for example, by heat exchange with steam or with another process stream in heater 61, or by any other suitable means for increasing the temperature of stream 54 within a desirable range. The temperature of both slurry streams 52 and 56 preferably is high enough to prevent crystallization of waxy components.

Slurry feedstream 56 comprises finely-divided solids (i.e., catalyst particles), solvent and waxy hydrocarbons, and it is fed to the solid/liquid separation unit 10. Solid/liquid separation unit 10 could comprise a settler, a hydrocyclone, a filter or any combination of two or more thereof. In preferred embodiments, solid/liquid separation unit 10 comprises a filter. Separation unit 10 generates hydrocarbon stream 55 which comprises at least a portion of waxy hydrocarbons and solvent from the slurry feedstream 56. Hydrocarbon stream 55 exits unit 10 and can be sent for subsequent refining and upgrading, for example for generating salable products such as diesel and/or naphtha and for recovering some of or all of the solvent for recycling to system 5.

In a preferred embodiment shown in FIG. 1, a plurality of filters 10, 20, 30 is placed in series to subject the slurry stream 52 to a multi-staged filtration process. In each stage of the multi-stage process, naphtha is added as a portion of a solvent feed 50 to the slurry feedstream at a point just prior to each filter, such that the diluted slurry stream passes through the filter, wherein it is separated into a liquid filtrate and a slurry retentate. The slurry retentate together with added naphtha provides the slurry feedstream to the next filter. At each point where solvent such as naphtha is added to the slurry stream, valves can regulate the amount of naphtha added. Preferably the amount (e.g., volume) of added naphtha can vary between about 10% and about 80% of the slurry volumetric flow rate, but preferably, the ratio of naphtha volumetric flow rate to slurry retentate volumetric flow rate can vary from 0.8:1 to 1.2:1. In each instance, the filtrates, e.g., streams 55, 65, and 75, comprising some of the waxy hydrocarbons and some of the naphtha are sent to a refining/upgrading unit for subsequent processing and recycling of the naphtha (as solvent feed 50) back to system 5. The filtration stages are set up in series so that substantially all of the waxy hydrocarbons have been removed from the catalyst exiting the last filter. Each filtrate stream from a filtering stage can be monitored with analyzers to determine the boiling point of a given filtrate. Generally, the boiling point of the filtrate will decrease with subsequent filtering stages, as the ratio of solvent to waxy hydrocarbons increases. Preferably, a number of filtering stages is used such that, as indicated by the boiling point of the last stage, the remaining waxy hydrocarbon and solvent in the slurry exiting the last filtering stage can be vaporized when contacted with a stripping gas in stripper 40 as described below. When naphtha is used as the solvent, the dilution/filtering process is complete when the filtrate preferably has a boiling point within the range of about 300° F. to about 420° F. (about 150-215° C.), and an effective number of filtering stages are employed to provide such a filtrate boiling point. The number of filters placed in series for a desired amount of hydrocarbon removal from the catalyst using a given solvent can be predetermined through computerized process simulation. In some embodiments, more than 90 percent of residual hydrocarbons are removed from the slurry feedstream 56 through the multi-staged filtration process. In other embodiments, more than 95 percent of residual hydrocarbons are removed from the slurry feedstream 56 through the multi-staged filtration process.

In FIG. 1, a simplified number (i.e., 3) of separation units is shown for demonstration purposes and to avoid visual redundancy in the diagram. In an embodiment for the removal of waxy hydrocarbons from a Fischer-Tropsch catalyst with naphtha as the solvent, a filtering process could comprise at least 2 stages in series, preferably at least 3 stages in series, more preferably at least about 5 filtering stages in series, still more preferably comprises at least about 10 filtering stages in series. In some embodiments, the number of filtering stages could equal or exceed 12.

Referring to FIG. 1, a retentate slurry 58 comprising the solid catalyst, solvent, and a reduced amount of waxy hydrocarbons exits separation unit 10 comprising a filter. The flow rate of retentate slurry 58 may be controlled by a valve (not shown) immediately downstream of separation unit 10. The retentate slurry 58 comprising catalyst, solvent, and waxy hydrocarbons is then combined with solvent stream 62, which is a slipstream (portion) of solvent feed 50. The flow rate of solvent stream 62 can be regulated by a valve (not shown) placed upstream of the combination point with retentate slurry 58. The solvent in solvent stream 62 further dilutes the hydrocarbons in retentate slurry 58, thereby further reducing the vaporization temperature of the resulting combination by removing some heavy waxy hydrocarbons from the solid (catalyst) surface and displacing them with lighter hydrocarbons into the hydrocarbon mixture so as to form a diluted slurry stream 64. Slurry stream 64 is then fed to separation unit 20 to be separated therein as described previously. Solid/liquid separation unit 20 preferably comprises a filter and separates from the diluted slurry stream 64 a portion of waxy hydrocarbons and some solvent as filtrate 65. Filtrate 65 can be sent for subsequent refining and upgrading. A retentate slurry stream 66 comprising solids (catalyst), solvent, and a reduced amount of waxy hydrocarbons exits separation unit 20. The flow rate of retentate slurry stream 66 can be controlled by a valve (not shown) immediately downstream of separation unit 20.

The retentate slurry stream 66 is further combined with solvent stream 68 (i.e., a portion of solvent feed 50) to form diluted slurry stream 74. The flow rate of solvent stream 68 may be regulated by a valve (not shown) placed upstream of the combination point with retentate slurry 66. The resulting diluted slurry stream 74 is fed to separation unit 30 and separated therein as described previously. Solid/liquid separation unit 30 preferably comprises a filter and separates a portion of waxy hydrocarbons and a portion of solvent as filtrate 75 which can be sent for subsequent refining and upgrading. Separation unit 30 also generates a retentate slurry 76 comprising the solids (catalyst), solvent, and a reduced amount of waxy hydrocarbons. Retentate slurry 76 exits separation unit 30 and its flow rate can be regulated by a valve (not shown) immediately downstream of separation unit 30.

Separation unit 10 comprising a filter, as well as each separation unit 20 or 30 comprising a filter in the subsequent separation stages, removes a percentage of the total volume of diluted slurry inside the filter as filtrate in the range of from about 3 to about 20% of the filter volume, preferably in a range of from about 3 to about 20% of the filter volume, and more preferably from about 8 to about 12% of the filter volume. The liquid stream comprising a waxy hydrocarbon and solvent mixture exiting one of the filtration units has a volumetric flow rate of about 3 to 50% of the total volumetric flow rate of the incoming slurry feedstream to said filtration unit. In some embodiments, the liquid stream exiting one of the filtration units has a volumetric flow rate of about 8 to 35% of the total volumetric flow rate of the incoming slurry feedstream to said filtration unit. In alternate embodiments, the liquid stream exiting one of the filtration units has a volumetric flow rate of about 3 to 20% of the total volumetric flow rate of the incoming slurry feedstream to said filtration unit. In yet some other embodiments, the liquid stream exiting one of the filtration units has a volumetric flow rate of about 8 to 12% of the total volumetric flow rate of the incoming slurry feedstream to said filtration unit.

As described previously, an additional number of dilution/filtration steps are performed until the boiling point of the filtrate exiting the last separation unit, e.g., stream 75 from separation unit 30, is within a predetermined range for the given solvent used to remove the hydrocarbons from the catalyst. Upon completion of the filtering process, substantially all of the waxy hydrocarbons have been removed from the solid catalyst, leaving at most only a residual amount that can be readily volatized and removed from the catalyst in the optional solvent stripper 40.

The wax-reduced retentate slurry 76 exiting the last separation unit 30 in the series should contain solid catalyst particles, solvent, and a small amount of residual waxy hydrocarbons. The amount of residual waxy hydrocarbons should be less than about 10% of the original amount in slurry feed 52; preferably between about 0.5 and 10%; more preferably between about 2 and 8%. Retentate slurry 76 is fed to the top of stripper 40 to remove the solvent and residual waxy hydrocarbons from the catalyst. A stripping gas 78, such as hydrogen, methane or other gaseous hydrocarbon, natural gas, nitrogen, steam such as superheated steam, a tail gas from a unit operation proximal to or within system 5, or any combination thereof, enters the bottom of stripper 40 and flows upward through the wax-reduced retentate slurry 76. The stripping gas 78 may contain small amounts of other gases, e.g., carbon dioxide and light hydrocarbons, but preferably does not contain any hydrocarbons of the type being removed from the catalyst. Preferably, the stripping gas 78 is supplied from a convenient source within the gas to liquids facility, such as incoming gas to the facility, refining off-gas, reactor tail gas such as Fischer-Tropsch reactor tail gas, hydroprocessing tail gas, or combinations thereof. Stripping gas 78 is preferably supplied at a temperature greater than the boiling point of the solvent used or greater than the end boiling point of the solvent if the solvent comprises a mixture. In an embodiment where the solvent is naphtha with an end boiling point typically around 350-370° F. (about 175-190° C.), the stripping gas 78 is supplied at a temperature from about 400° F. to about 550° F. (from about 200 to about 290° C.). Preferably, substantially all of the solvent and residual hydrocarbons are stripped off and exit the top of stripper 40 along with the stripping gas via an outlet stream 79.

The solid catalyst is collected and removed from the base of the stripper 40 via stream 80, and a valve (not shown) can regulate the catalyst flow. The catalyst 80 preferably is passed to a degasser 60 to remove any remaining gas. Pressurized inert gas stream 82, preferably nitrogen, is added to the degasser 60, thereby blanketing the catalyst with the inert gas to prevent the escape of any residual hydrocarbon gas to the atmosphere when the dry catalyst 84 is collected. The off-gas 79 from the stripper 40 (e.g., stripper gas, residual hydrocarbons, and solvent) and the off-gas 86 from the degasser 60 (e.g., any residual hydrocarbons and/or solvent and the inert gas) may be combined and sent via stream 88 for further processing, for example to a refining process to recover the solvent, the stripping gas and/or the inert gas. The dry catalyst 90 is collected and passed from the degasser 60 to a storage and/or transportation vessel for reclamation. The dry catalyst 90 exiting degasser 60 is warm typically at a temperature ranging from 250° F. to 400° F., preferably from 250° F. to 350° F., more preferably from 275° F. to 325° F. Preferably, the dry warm catalyst is collected into one of two or more hoppers (not illustrated). One hopper, when full, is moved aside for cooling while a second hopper is loaded with warm, freshly-degassed catalyst.

In the preferred embodiment of this invention, the entire process is pressure driven and therefore pumps are not required. Preferably, the initial system process operates at about 375 to about 475 psia.

In alternate embodiments of FIG. 1, slurry stream 52 may directly serve as slurry feedstream 56 to separation unit 10 if its catalyst content and its flowability are suitable to be fed directly to separation unit 10. If slurry stream 52 serving as feedstream 56 (without dilution with solvent stream 54) has a temperature below the desired temperature range, slurry stream 52 may be preheated, for example, by heat exchange with steam or/and any hot process stream, or by a preheating (radiant-heat furnace) unit.

FIG. 2 shows a method for providing slurry stream 52 in FIG. 1. FIG. 2 shows a Fischer-Tropsch process 100 comprising Fischer-Tropsch reactor 110 and catalyst-hydrocarbon separation unit 115. Fischer-Tropsch reactor 110 preferably is a slurry bed reactor; however, other types of reactors are also envisioned. Slurry bed reactors are known in the art and are also referred to as “slurry reactors” or “slurry bubble columns.” Fischer-Tropsch reactor 110 as a slurry bed reactor operates by dispersing solid particles of a Fischer-Tropsch catalyst in a liquid inside a reactor vessel, wherein the mixture of solid catalyst particles and the liquid forms a slurry. The slurry in reactor 110, without gas hold-up, typically comprises between 5 percent by weight (wt %) and about 40 wt % of solid catalyst particles in a liquid. The liquid in the slurry typically comprises a hydrocarbonaceous liquid, preferably comprises a mixture of hydrocarbonaceous compounds, more preferably comprises hydrocarbon products synthesized in said Fischer-Tropsch reactor 110.

A feed stream 120 comprising hydrogen (H₂) and carbon monoxide (CO) (the mixture thereof typically called synthesis gas) is fed near or at the bottom of Fischer-Tropsch reactor 110 through a gas distributor (not shown), thereby producing gas bubbles. As the gas bubbles rise through the reactor, the H₂ and CO reactants are absorbed into the liquid and diffuse to the Fischer-Tropsch catalyst where they are converted to hydrocarbon liquids, hydrocarbon gases, and water. At least a portion of the hydrocarbon gases and the water enter the gas bubbles as they rise to the top of Fischer-Tropsch reactor 110 where they exit the reactor vessel via overhead gas stream 122. Fischer-Tropsch synthesis can produce a broad range of hydrocarbon products with carbon numbers ranging from 1 up to about 200, the majority of which are commonly referred to as waxes, comprised primarily of paraffins, some olefins, and some oxygenates (oxygen-containing hydrocarbonaceous compounds). At least a small portion of Fischer-Tropsch waxy hydrocarbons having greater than about 10 carbon atoms cannot be completely separated from the slurry using normal solid/liquid separation techniques, and these waxy hydrocarbons are typically part of the slurry liquid within reactor 110. Overhead gas stream 122 from reactor 110 may be combined with other off-gases or tail gases from other units, e.g., stream 88 from FIG. 1, to form overall tail gas 125. Overhead gas stream 122 and/or overall tail gas 125 may be sent to a gas plant (not shown) for further processing for at least in part recycling some unconverted reactants to various processes, including reactor 110 and other Fischer-Tropsch reactors.

At least a portion of the three-phase gas-expanded slurry comprising the Fischer-Tropsch catalyst and hydrocarbons and entrapped gas exits the Fischer-Tropsch reactor 110 as reactor slurry stream 130 at an exit point 131 typically, although not necessarily, in the top half of the reactor vessel and enters an external slurry circulation loop 128. The external slurry circulation loop 128 starts at reactor outlet 131 via stream 130, comprises a degasser 140 and a catalyst-hydrocarbon separation unit 115, and returns at reactor inlet 151 via stream 150. Since reactor slurry stream 130 typically comprises entrapped gas, reactor slurry stream 130 is passed through degasser 140 to separate entrapped gas from the slurry and form a degassed slurry stream 132 and degasser gas effluent 143. The degassed slurry stream 132 is typically a two-phase slurry containing catalyst particles and liquid waxy hydrocarbons.

Degassed slurry stream 132 is further passed through catalyst-hydrocarbon separation unit 115 so as to form hydrocarbon product stream 145 and solid-enriched slurry stream 144. Catalyst-hydrocarbon separation unit 115 can be any solid-liquid separation system, which can provide a liquid product stream (i.e., 145) which is catalyst-lean or substantially free of catalyst solid and a catalyst-rich slurry stream (i.e., 144). Catalyst-hydrocarbon separation unit 115 may employ one or more solid-liquid separation techniques such as filtration, decantation, sedimentation, centrifugation, magnetic separation, or any combination thereof. Catalyst-hydrocarbon separation unit 115 could comprise a settler, a filter, a hydrocyclone, a centrifuge, a magnetic separation unit, any plurality thereof, or any combination thereof. In preferred embodiments, catalyst-hydrocarbon separation unit 115 comprises a settler; a filter, any plurality thereof, or any combination thereof. In some embodiments, catalyst-hydrocarbon separation unit 115 comprises a settler and a filter operated in series. Preferably, all separation units in the catalyst recovery system 5 of FIG. 1 comprise the same type of separation units as separation unit 115 used in the external slurry circulation loop 128 of FIG. 2. In some embodiments, the catalyst-hydrocarbon separation unit 115 may also generate an optional gas effluent 148. Gas effluent 143 from degasser 140, overhead gas stream 122 from reactor 110, and optional gas effluent 148 from unit 115, may be combined to form overall tail gas 125.

Under typical reactor operating conditions, the catalyst-enriched slurry stream 144 would be recycled almost entirely to Fischer-Tropsch reactor 110 via line 150. However, when it is desired to remove all of or a portion of the catalyst-enriched slurry stream 144 for catalyst reclamation, the slurry stream 144 is either diverted either in totality (not shown) or partially to the catalyst recovery system 5 of FIG. 1. A portion 150 (i.e., a slipstream) of solid-enriched slurry stream 144 can be recycled to reactor 110, while another portion 152 of solid-enriched slurry stream 144 can provide slurry feedstream 56 or slurry stream 52 to the catalyst recovery system 5 as shown in FIG. 1. Further, when the slurry volume in Fischer-Tropsch reactor 110 declines to the point where the slurry level in reactor 110 is below the exit point 131 of slurry stream 130, the slurry inside reactor 110 may be allowed to exit Fischer-Tropsch reactor 110 from line 150 in reverse direction (not shown) to provide, in part or in totality, the slurry stream 52 or alternatively the slurry feedstream 56 to the catalyst recovery system 5 of FIG. 1.

Referring again to FIG. 2, Fischer-Tropsch product stream 145 can be fed to a fractionator 155 so as to separate its hydrocarbon components based on their boiling range. Hydrocarbon product stream 145 may comprise small amounts of solids which may be further removed by a polishing filter (not shown) prior to being sent to fractionator 155. Several fractions of different boiling ranges exit fractionator 155. Some of the fractions comprise at least in part a light gas effluent 158, a naphtha stream 160 and a middle distillate stream 162. Middle distillate stream 162 may comprise diesel or kerosene. Either of the naphtha stream 160 and middle distillate stream 162 or any combination of both may form the solvent feed 50 for the catalyst recovery system 5 of FIG. 1.

In an embodiment, a valve regulating the flow of slurry stream 58 immediately downstream of unit 10 can be also used to regulate the flow of slurry from external slurry circulation loop 128 from the reactor system 100. Additionally, when catalyst recovery system 5 is not being utilized, block valves in the line connecting slurry stream 152 from reactor system 100 to slurry stream 52 and/or slurry feedstream 56 of catalyst recovery system 5 may be closed so as to isolate the recovery system 5, for example to purge the facility with a cleaning fluid.

Although not illustrated in FIG. 2, a yet alternate method for providing slurry feedstream 56 or a portion of slurry feedstream 56 can be achieved by unloading the content of Fischer-Tropsch reactor 110, either partially or in totality. In this embodiment, Fischer-Tropsch reactor 110 preferably comprises a slurry bubble column reactor, said reactor containing a slurry comprising a solid catalyst and a molten waxy hydrocarbon liquid, wherein the solid catalyst is at least partially deactivated and is dispersed in the molten waxy hydrocarbon liquid. The Fischer-Tropsch reactor 110, when operating, is typically at a reaction temperature between 160° C. and 300° C., preferably between 190° C. and 260° C., more preferably between 205° C. to 230° C. When the reactor shutdown is commenced, the composition of gas feedstream 120 may be modified such as by substituting one or both of the reactant gases by another unreactive gas like methane, natural gas or other suitable gaseous stream, or by removing one of the reactant gases, so as to stop the reaction in Fischer-Tropsch reactor 110. Alternatively or in addition, the slurry within reactor 110 may be cooled from the reaction temperature to a lower temperature so as to stop the reaction and make it more convenient to start the catalyst unloading process. The cooling may be intermittent or continuous. The cooling may commence before, during or after modifying the composition of the gas feedstream 120. The cooling of the slurry within reactor 110 however could cause solidification of some of the waxy hydrocarbon components. Therefore, alternatively or in addition, a lighter diluting hydrocarbon liquid (i.e., lighter in boiling range than molten waxy hydrocarbon liquid) may be added, either periodically or continuously, to reactor 110 so as to gradually lower the content in molten waxy hydrocarbons in the slurry contained in the reactor 110, while the slurry stream 130 continuously goes through external slurry circulation loop 135 and get separated in the liquid-solid separation unit 115. With the addition of the lighter diluting hydrocarbon liquid to the slurry in reactor 110, the cooling of the slurry may be excluded if the reaction in the slurry has been stopped by removing at least one or both of the reactant gases. The cooling of the slurry is preferably performed. The reactor may comprise internal coils or tubes disposed within the slurry, and the cooling of the slurry is performed by passing a cooling medium through the internal coils or tubes to remove some of the heat from the slurry and thereby decreasing its temperature. Cooling may be done before, during or after the addition of the lighter diluting hydrocarbon liquid. Cooling may be done after modification of gas feed composition but before the addition of the lighter diluting hydrocarbon liquid. The waxy hydrocarbon liquid in hydrocarbon product stream 145 and in solid-enriched slurry stream 148 is gradually replaced by the lighter diluting hydrocarbon liquid. The solid-enriched slurry stream 148 preferably in its entirety is recycled to reactor 110 such that a majority of the wax in the slurry present in reactor 110 is replaced by the introduced diluting hydrocarbon liquid and up to a point at which the slurry can achieve an acceptable temperature without causing solidification of the newly-combined wax-reduced slurry. The acceptable temperature may range from ambient temperature to about 160° C., preferably from ambient temperature to about 120° C. Once the slurry within reactor 110 achieves the acceptable temperature, the addition of diluting hydrocarbon liquid to the reactor vessel can be stopped. After the acceptable temperature is reached, the newly-combined wax-reduced slurry in reactor 110 can then be withdrawn via stream 130 and/or stream 150 (in reverse flow) so as to provide, in part or in totality, the slurry feedstream 56 to the catalyst recovery facility 10 of FIG. 1, which comprises a liquid-solid separation unit. The newly-combined wax-reduced slurry in reactor 110, either as a portion of the reactor content or as the totality of reactor content, can be sent via a wax-reduced slurry stream to a holding vessel (or more than one holding vessels) prior to being fed to the catalyst recovery facility 10 of FIG. 1. The holding vessel may comprise heating so as to maintain the temperature of wax-reduced slurry at or above about the acceptable temperature without causing solidification of the wax-reduced slurry. The optional holding vessel may be heated to keep in a molten state wax hydrocarbons present in the wax-reduced slurry as well as the wax hydrocarbons onto and/or into the solid catalyst. In addition, the wax-reduced slurry contained in the holding vessel is preferably agitated in order to keep the solid catalyst dispersed in the wax-reduced slurry so as to prevent the solid catalyst from settling to the bottom of the holding vessel. The agitation may be provided by supplying a fluidization gas to the bottom of the vessel(s) and/or by continuously circulating a portion of the vessel content (so as to create fluid turbulence within the vessel), for example with a re-circulating pump (not shown). The holding vessel may further comprise a fluidization gas inlet located at or near the bottom of said vessel(s) and configured to pass the fluidization gas through the wax-reduced slurry disposed in the holding vessel. The fluidization gas should have a gas velocity sufficient to maintain the catalyst solids in suspension in the wax-reduced slurry contained in the holding vessel. It should be noted that, while the diluting lighter hydrocarbon liquid is continually added to reactor 110 and the portion 150 of solid-enriched slurry stream 148 is returned to reactor 110, product stream 145 comprising less and less waxy hydrocarbons (as stream 145 is gradually enriched in lighter diluting hydrocarbon liquid) is continuously removed from reactor 110 so as to prevent reactor liquid overflow.

In preferred embodiments, the lighter diluting hydrocarbon liquid has a higher boiling range than solvent feed 50 of FIG. 1. In more preferred embodiments, the lighter diluting hydrocarbon liquid added to reactor 110 of FIG. 2 comprises a hydrocarbon mixture within the diesel, kerosene, or lubricating oil boiling range (as a non-limiting example, a portion of middle distillate fraction 162), while solvent feed 50 of FIG. 1 comprises a hydrocarbon mixture within the naphtha boiling range (as a non-limiting example, a portion of fraction 160 shown in FIG. 2). The solid-enriched slurry stream 144 preferably in its entirety is recycled to reactor 110 such that a majority of the wax in the slurry contained in reactor 110 is replaced by the diluting hydrocarbon liquid up to a point at which the slurry can achieve an acceptable temperature without causing solidification of the newly-combined wax-reduced slurry. The acceptable temperature may range from ambient temperature to about 160° C., preferably from ambient temperature to about 120° C. The cooler wax-reduced slurry in reactor 110 can then be withdrawn via stream 130 and/or stream 150 (in reverse flow) so as to provide, in part or in totality, the slurry feedstream 56 (or alternatively slurry stream 52) to the catalyst recovery system 5 of FIG. 1. It should be noted that while the diluting lighter hydrocarbon liquid is continually added to reactor 110 and that the catalyst-enriched slurry 150 is returned to reactor 110, product stream 145 comprising less and less waxy hydrocarbons (as stream 145 is gradually enriched in lighter diluting hydrocarbon liquid) is continuously removed from reactor 110 so as to prevent reactor liquid overflow. By unloading the reactor slurry in this manner, the separation system 115 of FIG. 2 could be considered as performing the primary duties of the first solid-liquid separation unit 10 of FIG. 1. Hence there may be a need of only a few additional downstream solid/liquid separation units to achieve the desired removal of residual hydrocarbons.

FIG. 3 illustrates a liquid-liquid extraction/separation unit 210, which could be used in solid/liquid separation units 10, 20, and 30 of FIG. 1 and/or in catalyst-hydrocarbon separation unit 115 of FIG. 2. Extraction/separation unit 210 contains, in its upper end, a separation zone characterized by filtration units 220 and, in its lower end, a liquid-liquid extraction zone characterized by contacting plates 225. A slurry stream 230 (e.g., the slurry stream 52 as described in FIG. 1 or the slurry stream 132 as described in FIG. 2) is fed to the upper end of extraction/separation unit 210 so that solid particles migrate downward through the separation zone, while an extraction medium 240 (similar to solvent stream 54 as described in FIG. 1) is fed in the lower end of extraction/separation unit 210 preferably below the location of contacting plates 225 within the extraction zone.

Extraction/separation unit 210 should be designed to effect good liquid-liquid extraction of the slurry stream 230 by extraction medium 240. The catalyst particles and extraction medium 240 flow mainly in a counter-current manner in the extraction zone wherein the presence of contacting plates 225 should promote good contact between downward slurry flow and upward flow of extraction medium. The contact should be effective such that extraction medium 240 extracts some of the waxy hydrocarbon liquid from the slurry, particularly some of the residual hydrocarbons from the solid catalyst. The main purpose of the extraction medium 240 is to displace waxy hydrocarbon liquid from slurry stream 230 to extraction medium so as to form a spent extraction medium (i.e., containing waxy hydrocarbons) which moves upwards into the separation zone of unit 210 and passes through filtration units 220. Extraction medium 240 preferably comprises an extraction liquid selected from the group consisting of a diesel, a naphtha, a gasoline, a kerosene, a gas oil, a heating oil, a solvent, and any combination thereof. Extraction medium 240 more preferably comprises a light hydrocarbon liquid such as naphtha and/or diesel, and most preferably, a synthetic light hydrocarbon liquid such as naphtha and/or diesel derived from a Fischer-Tropsch synthesis. A filtrate 250 exits filtration units 220, e.g., streams 55, 65, or 75 in FIG. 1, wherein filtrate 250 should be substantially free of solids and comprises said spent extraction medium (i.e., hydrocarbon-enriched extraction medium), while a retentate (not shown) exiting filtration units 220 is retained within unit 210. The extracted particles dispersed in a liquid containing some extraction medium exit unit 210 via slurry stream 256. Slurry stream 256 could provide, in part or in totality, any of the slurry feedstreams 52, 58, 66, or 76 of the catalyst recovery system 5 of FIG. 1.

FIGS. 4a and 4b illustrate a rotary filtration unit 310, which could be used in solid/liquid separation units 10, 20, and 30 of FIG. 1 and/or in catalyst-hydrocarbon separation unit 115 of FIG. 2. Rotary filtration unit 310 preferably comprises a rotary drum vacuum filter as shown as 315 in FIG. 4b. A rotary drum vacuum filter 315 can be operated in a continuous manner, wherein a slurry stream 330 (which may be the slurry stream 52 as described in FIG. 1 or the slurry stream 132 as described in FIG. 2) is separated by a porous substrate, such as cloth or other suitable media, which rotates through the slurry. A vacuum is applied to its inner surface to cause the solids to accumulate on its external surface and form a cake or solid layer through which a liquid hydrocarbon filtrate 350 is drawn. A vacuum is applied to its inner surface while the rotary drum vacuum filter 315 rotates so as to cause the solids from the slurry to accumulate on its external surface as a cake or solid layer through which a liquid hydrocarbon filtrate 350 is drawn. Rotary filtration unit 310 should be designed to effect the production of a stream enriched in catalyst particles. Additionally, rotary filtration unit 310 should be designed to clean the solid catalyst deposited on the filter medium by contacting the catalyst-containing cake with washing medium 340 (which may be the same as or similar to solvent feed 50 or solvent streams 54, 62, 68). Washing medium 340 preferably comprises a light hydrocarbon liquid such as naphtha and/or diesel, preferably Fischer-Tropsch naphtha, and its main purpose is to remove some of the residual hydrocarbons from the solid catalyst surface by displacement of the residual hydrocarbons from the catalyst surface to the washing medium. As the rotary drum vacuum filter 315 rotates while partially submerged in the slurry, vacuum draws the liquid filtrate 350 (comprising the washing medium with the removed residual waxy hydrocarbon liquid) through the catalyst cake and filter medium on the drum. The filtrate 350 flows through internal pipes(s) (not shown) before exiting rotary drum vacuum filter 315. The rotary drum vacuum filter 315 can discharge its filtered and washed catalyst cake by means of several discharge arrangements, such as a scraper 360 (as shown in FIG. 4b), belt, or roll (not shown). The operation is typically cyclic and continuous, which each revolution of the drum 315 comprises of cake formation, cake washing with a liquid washing medium 340, optional drying, and cake discharge, so that filtrate 350 (catalyst-free) and a catalyst-enriched slurry 356 exit rotary filtration unit 310. Slurry stream 356 can provide, in part or in totality, the slurry feedstream 52, 58, 66, or 76 of the catalyst recovery system 5 of FIG. 1.

In a preferred commercial embodiment, the catalyst recovery processes and systems according to the present invention may be operated in a continuous mode, a batch mode, and combinations thereof. In continuous mode of separation in FIG. 2, a desired percentage of catalyst is continuously removed from the external slurry circulation loop 128 during operation of the Fischer-Tropsch reactor 110, and a like percentage of fresh catalyst is added to the reactor. While in continuous mode, a diverted portion 152 of catalyst-enriched stream 144 enters stream 52 of FIG. 1 for processing as described previously. In batch mode, flow of stream 144 can be totally diverted during shut down of Fischer-Tropsch reactor 110 to change out substantially all the catalyst either into stream 52 or in stream 56 (if a diluting stream is added during shut-down and cool-down of the reactor 110) for processing.

In an embodiment, at start up of a given Fischer-Tropsch reactor the catalyst is fresh and therefore the catalyst recovery system 5 employing a multi-staged wax displacement would not be utilized for that particular reactor for an initial time period. After operating the Fischer-Tropsch reactor for a period of time, it may become desirable to begin the continual or continuous replacement of a portion of the catalyst inventory. A desired amount of fresh catalyst is added while an equivalent amount of spent catalyst is removed and sent to the catalyst recovery system 5 for its recovery. An intermediate storage vessel (not shown) for storage of a slurry comprising spent solid catalyst may be disposed between Fischer-Tropsch reactor 22 and facility 50 for minimizing reactor downtime. After the Fischer-Tropsch reactor has been in operation for an additional period of time, the bulk of the catalyst in the slurry bed reactor will become deactivated. At this point, the reactor is shut down and all of the spent catalyst in the form of a slurry is removed and sent to the catalyst recovery system 5 employing the multi-staged wax displacement for its recovery in a batch process. The reactor is subsequently loaded with fresh catalyst, and the Fischer-Tropsch process is started up again.

In some embodiments (not illustrated), a single catalyst recovery facility 5 can be manifolded to multiple synthesis reactors 110, for example Fischer-Tropsch reactors. Therefore, overall plant operation can be continuous by scheduling only a single Fischer-Tropsch reactor to be shut down at a time for catalyst change out and recovery while all others are in operation. Alternatively, continuous slurry streams comprising spent catalyst from each of the operating reactors can provide continually the slurry stream 52 to the single catalyst recovery facility 5. Operating in this manner would improve the economics for and the use of a catalyst recovery system 5 according to the present invention.

In some alternate embodiments (not illustrated) of FIG. 1, solvent streams 54, 62 and 68 comprise different compositions, i.e., are not slip streams of a single solvent feed (such as solvent feed 50). For example, the boiling ranges of the solvent streams 54, 62 and 68 may differ. It is also envisioned that, for example, a succession of lighter and lighter solvents could be employed in the multi-staged wax displacement system comprising solid-liquid separation units 10, 20 and 30, such that the last solvent stream used (i.e., solvent stream 68) has a lighter boiling point range than the first solvent stream used (i.e., solvent stream 54). In some embodiments, solvent stream 54 may be a synthetic diesel such as middle distillate 162 from fractionator 155 of FIG. 2 or a diesel fraction therefrom; solvent stream 58 may be a synthetic jet fuel or kerosene such as middle distillate 162 from fractionator 155 of FIG. 2 or a kerosene fraction therefrom; and solvent stream 68 may be a synthetic naphtha such as naphtha 160 from fractionator 155 of FIG. 2 or a naphtha fraction therefrom.

In another alternative embodiment, the Fischer-Tropsch process occurs in a fixed bed reactor. The catalyst recovery system as described herein could be used on the spent Fischer-Tropsch fixed bed reactor catalyst provided that additional means are employed to remove the spent catalyst from the reactor. For example, upon freeing the catalyst within the bed, an available solvent such as naphtha could be pumped through nozzles to wash the spent catalyst slurry out from the Fischer-Tropsch fixed bed reactor into the catalyst recovery system 5 as described previously. Additionally, the catalyst recovery process would need to have its capacity adjusted accordingly to take into account the change in catalyst structure needed to accommodate fixed bed reactors, as the catalyst particle size is normally bigger (typically greater than 0.5 mm) for a fixed bed reactor versus a slurry bed reactor (typically less than greater than 0.25 mm with a weight average particle size between about 50 and about 100 microns, preferably between about 60 and about 90 microns). Initial system pressure would no longer drive the process so pumps or other suitable transport means would preferably be utilized to supply the slurry feedstock to catalyst recovery system 5.

The separation process described in the embodiments above may be used to remove residual hydrocarbons from any suitable spent catalyst. In an embodiment shown in the figures, the catalyst described above is utilized in a hydrocarbon liquid synthesis process, preferably a Fischer-Tropsch process, to promote the conversion of CO and H₂ to one or more hydrocarbons.

FIG. 2 depicts a hydrocarbon liquid synthesis process, preferably a Fischer-Tropsch process, wherein the spent catalyst from the synthesis reactor 110 provides the feed for the catalyst recovery system 5 of the present invention. In a preferred embodiment shown in FIG. 2, at least a portion of the syngas feed 120 is preferably provided by one syngas reactor which comprises a partial oxidation (POX) reactor. In a more preferred embodiment, the POX reactor comprises a catalyst. A hydrocarbon feedstream comprising one or more alkanes, e.g., methane or natural gas, is fed to a partial oxidation (POX) reactor for conversion to syngas. The hydrocarbon feedstream may be a natural gas stream comprising alkanes such as methane, ethane, and propane. Alternatively, hydrocarbon feedstream may be a stream recovered from a gas plant (not shown) used to process natural gas into different fractions. Methane or other suitable hydrocarbon feedstreams (hydrocarbons with four carbons or less) are also readily available from a variety of other sources such as higher chain hydrocarbon liquids, coal, coke, hydrocarbon gases, etc., all of which are clearly known in the art. Preferably, the hydrocarbon feedstream to the POX reactor comprises essentially the methane fraction recovered from a gas plant processing natural gas. An oxygen-containing gas (e.g., pure oxygen, oxygen diluted with an inert gas, air, oxygen-enriched air, and so forth) is combined with the hydrocarbon feedstream and passed under conversion promoting conditions through the POX reactor so as to form a synthesis gas. The POX reactor is preferably a short contact time reactor (SCTR), e.g., a millisecond contact time reactor. The partial oxidation of the methane to syngas proceeds by the following exothermic reaction:
2CH₄+O₂→2CO+4H₂

The conversion promoting conditions preferably includes a partial oxidation catalyst disposed within the POX reactor. The POX reactor contains any suitable catalyst for promoting the conversion of hydrocarbon gas to syngas. The POX catalyst comprises a wide range of catalytically active components, e.g., palladium, platinum, rhodium, iridium, osmium, ruthenium, nickel, chromium, cobalt, cerium, lanthanum, and mixtures thereof. A portion of or the totality of syngas stream 120 comprising H₂ and CO is recovered from the POX reactor.

Within the POX reactor, hydrocarbon feedstream comprising methane is contacted with the POX catalyst in a reaction zone that is maintained at conversion-promoting conditions effective to produce H₂ and CO. Preferably, the POX reactor is operated at such conditions to avoid the formation of unwanted by-products.

The gas hourly space velocity of the feedstream in the POX reactor can vary widely. Space velocities for the syngas production process via partial oxidation, stated as gas hourly space velocity (GHSV), are in the range of about 20,000 to about 100,000,000 hr⁻¹, more preferably of about 100,000 to about 10,000,000 hr⁻¹, still more preferably of about 100,000 to about 4,000,000 hr⁻¹, yet still more preferably of about 400,000 to about 700,000 hr⁻¹. Although for ease in comparison with prior art systems space velocities at standard conditions have been used to describe the present invention, it is well recognized in the art that residence time is the inverse of space velocity and that the disclosure of high space velocities corresponds to low residence times on the catalyst. “Space velocity,” as that term is customarily used in chemical process descriptions, is typically expressed as volumetric gas hourly space velocity in units of hr⁻¹. Under these operating conditions a flow rate of reactant gases is maintained sufficient to ensure a residence or dwell time of each portion of reactant gas mixture in contact with the catalyst of no more than 200 milliseconds, preferably less than 50 milliseconds, and still more preferably less than 20 milliseconds. A contact time less than 10 milliseconds is highly preferred. The duration or degree of contact is preferably regulated so as to produce a favorable balance between competing reactions and to produce sufficient heat to maintain the catalyst at the desired temperature. In order to obtain the desired high space velocities, the process is operated at atmospheric or superatmospheric pressures. The pressures may be in the range of about 100 kPa to about 32,000 kPa (about 1-320 atm), preferably from about 200 kPa to about 10,000 kPa (about 2-100 atm); more preferably from about 200 kPa to about 5,000 kPa (about 2-50 atm). The POX reactor which comprises a catalyst (CPOX) is preferably operated at a temperature in the range of about 350° C. to about 2,000° C. More preferably, the temperature is maintained in the range 400° C.-2,000° C., as measured at the CPOX reactor outlet. Additional description for operating a CPOX reactor is disclosed in co-owned U.S. Pat. Nos. 6,402,989; 6,409,940; 6,461,539; 6,630,078; 6,635,191; and US published patent application 2002-0115730, each of which is incorporated herein by reference in its entirety.

In alternative embodiments, POX reactor may be replaced with or supplemented with by other syngas production units capable of converting a methanehydrocarbon gas feedstream (such as methane, ethane, or natural gas) to syngas, such as a steam reformer, a dry reformer and/or an auto-thermal reformer. Dry reforming entails reacting light hydrocarbons and carbon dioxide. Steam reforming (SR) entails endothermically reacting light hydrocarbons and steam over a POX catalyst contained within a plurality of externally heated tubes mounted in a furnace. Auto-thermal reforming (ATR) employs a combination of steam reforming and partial oxidation, i.e., reacting light hydrocarbons with oxygen and steam. More particularly, the endothermic heat required for the steam reforming reaction is obtained from the exothermic partial oxidation reaction. Suitable conditions for operating a steam reforming reactor and a dry reforming reactor are disclosed in V. R. Choudhary et al., in Catalysis Letters (1995) vol. 32, pp. 387-390; S. S. Bharadwaj & L. D. Schmidt in Fuel Process. Technol. (1995), vol. 42, pp. 109-127; and Y. H. Hu & E. Ruckenstein, in Catalysis Reviews—Science and Engineering (2002), vol. 44(3), pp. 423-453, each of which is incorporated herein by reference in its entirety.

The syngas stream 120 recovered from a syngas synthesis unit such as a POX reactor is fed to a synthesis reactor wherein the syngas is converted to a hydrocarbon liquid product such as Fischer-Tropsch products comprising mainly paraffins with some olefins and oxygenates liquids and/or alcohol, typically by contact with a synthesis catalyst. In a preferred embodiment shown in FIG. 2, the synthesis reactor 110 is a Fischer-Tropsch reactor containing any suitable Fischer-Tropsch catalyst for promoting the conversion of syngas to hydrocarbon liquids. In an alternative embodiment, synthesis reactor 110 is an alcohol synthesis reactor containing any suitable catalyst for promoting the conversion of syngas to one or more alcohols, preferably methanol

In the Fischer-Tropsch reactor embodiment, the syngas stream 120 is fed to a Fischer-Tropsch reactor 110 containing the Fischer-Tropsch catalyst to be recovered by the present invention, i.e., a metal catalyst activated by the partial reduction of metal oxide present on a catalyst support. The feed gases charged to the process of the invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, auto-thermal reforming, dry reforming, advanced gas heated reforming, partial oxidation, catalytic partial oxidation, or other processes known in the art. Alternatively, the H₂/CO mixtures can be obtained from biomass and/or from coal by gasification. In addition, the feed gases can comprise off-gas recycle from the present or another Fischer-Tropsch process. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than about 0.5:1 (e.g., from about 0.67 to about 2.5). Preferably, when cobalt, nickel, and/or ruthenium catalysts are used, the feed gas stream contains hydrogen and carbon monoxide in a molar ratio of about 1.4:1 to about 2.3:1. Preferably, when iron catalysts are used, the feed gas stream contains hydrogen and carbon monoxide in a molar ratio of about 1.4:1 to about 2.2:1. The feed gas may also contain carbon dioxide. The feed gas stream should not contain, but at a very low concentration, compounds or elements that have a deleterious effect on the catalyst, such as poisons. For example, the feed gas may need to be pretreated to ensure that it contains no or alternatively very low concentrations of sulfur or nitrogen compounds such as hydrogen sulfide, ammonia and carbonyl sulfides.

During the Fischer-Tropsch process in which syngas stream 120 is fed to a Fischer-Tropsch reactor 110, the reaction zone contained in Fischer-Tropsch reactor 110 is maintained at conversion-promoting conditions effective to produce the desired hydrocarbon liquids. The Fischer-Tropsch process is typically run in a continuous mode. In this mode, the gas hourly space velocity through the reaction zone typically may range from about 50 hr⁻¹ to about 10,000 hr⁻¹, preferably from about 300 hr⁻¹ to about 2,000 hr⁻¹. The gas hourly space velocity is defined as the volume of reactants per time per reaction zone volume. The volume of reactant gases is at standard conditions of pressure (101 kPa) and temperature (0° C.). The reaction zone volume is defined by the portion of the reaction vessel volume where reaction takes place and which is occupied by a gaseous phase comprising reactants, products and/or inerts; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions at temperatures from about 190° C. to about 260° C., more preferably from about 205° C. to about 230° C. The reaction zone pressure is typically in the range of from about 80 psia (552 kPa) to about 1000 psia (6895 kPa), more preferably from about 80 psia (552 kPa) to about 800 psia (5515 kPa), and still more preferably, from about 140 psia (965 kPa) to about 750 psia (5170 kPa). Most preferably, the reaction zone pressure is from about 250 psia (1720 kPa) to about 650 psia (4480 kPa).

Any suitable reactor configuration that allows contact between the syngas and the catalyst may be employed for Fischer-Tropsch reactor 110. The feed gas is contacted with the catalyst in a reaction zone. Mechanical arrangements of conventional design may be employed as the reaction zone including, for example, fixed bed, fluidized bed, slurry bubble column or ebulliating bed reactors, among others. Accordingly, the preferred size and physical form of the catalyst particles may vary depending on the reactor in which they are to be used. Most preferably, Fischer-Tropsch reactor 110 comprises a slurry bubble column reactor loaded with solid catalyst particles comprising cobalt and/or ruthenium with optionally promoters. The solid catalyst particles may have a size varying from sub-micron up to about 250 microns, but preferably 90 percent by weight of the particles should have a size between about 10 and 150 microns. The solid catalyst particles should have a weight average size between about 30 microns and 150 microns, preferably between about 40 microns and 100 microns, more preferably between about 60 microns and 90 microns.

Fischer-Tropsch catalysts are well known in the art and generally comprise a catalytically active metal, a promoter and optionally a support structure. The most common catalytic metals are Group 8, 9 and 10 metals of the Periodic Table (new IUPAC Notation), such as cobalt, nickel, ruthenium, and iron or mixtures thereof. The preferred metals used in Fischer-Tropsch catalysts with respect to the present invention are cobalt, iron and/or ruthenium, however, this invention is not limited to these metals or the Fischer-Tropsch reaction. Other suitable catalytic metals include Groups 8, 9 and 10 metals. The promoters and support material are not critical to the present invention and may be comprised, if at all, by any composition known and used in the art. Promoters suitable for Fischer-Tropsch synthesis may comprise at least one metal from Group 1, 7, 8, 9, 10, 11, and 13. When the catalytic metal is cobalt, the promoter is preferably selected from the group consisting of ruthenium (Ru), platinum (Pt), palladium (Pd), rhenium (Re), boron (B), silver (Ag), and combinations thereof. When the catalytic metal is iron, the promoter is preferably selected from the group consisting of lithium (Li), copper (Cu), potassium (K), silver (Ag), manganese (Mn), sodium (Na), and combinations thereof The preferred support composition when used preferably comprises an inorganic oxide selected from the group consisting of alumina, silica, titania, zirconia and mixtures thereof. The inorganic oxide is preferably stabilized by the use of a structural promoter or stabilizer, so as to confer hydrothermal resistance to the support and the catalyst made therefrom.

In preferred embodiments, Fischer-Tropsch process 100 comprises one or more hydrocarbon synthesis reactors and each reactor comprises a slurry bubble column operated with particles of a cobalt catalyst.

In a slurry-bubble reactor, the Fischer-Tropsch catalyst particles are suspended in a liquid, e.g., molten hydrocarbon wax, by the motion of bubbles of syngas sparged into the bottom of the reactor. As the gas bubbles rise through the reactor, the syngas is absorbed into the liquid where it diffuses to the catalyst for conversion to hydrocarbons. Gaseous products enter the gas bubbles and are collected at the top of the reactor. Liquid products are recovered from the suspended liquid using different techniques such as filtration, settling, hydrocyclones, and magnetic techniques. Cooling coils immersed in the slurry remove heat generated by the reaction.

Alternatively, the Fischer-Tropsch reactor can be a fixed bed reactor in which the Fischer-Tropsch catalyst bed is held in a fixed arrangement that is maintained within the reactor vessel. The syngas flowing through the Fischer-Tropsch catalyst fixed bed reactor vessel contacts the Fischer-Tropsch catalyst contained in the fixed bed. The reaction heat is typically removed by passing a cooling medium within cooling tubes disposed within the vessel that contains the fixed catalytic bed.

Referring to FIG. 2, H₂ and CO combine in a polymerization-like fashion in Fischer-Tropsch reactor 110 to form hydrocarbon compounds having varying numbers of carbon atoms. The hydrocarbon compounds are typically separated in a fractionator illustrated as distillation unit 155 by boiling point into different fractions. Some of the fractions comprise at least in part a light gas effluent 158, a naphtha stream 160 and a middle distillate stream 162, with each stream having the majority of the hydrocarbons falling within a given range of carbon atoms. Generally, the higher the boiling point, the higher the wax content of the stream. Solvent feed 50 of FIG. 1 may be one of these fractions 160 or 162, or a combination of these two fractions. The naphtha stream 160 typically comprises liquid synthetic paraffins having about five to about nine or ten carbon atoms. The middle distillate stream 162 typically comprises liquid synthetic paraffins having about nine to about 20 or 21 carbon atoms. A gas fraction which is typically produced by Fischer-Tropsch reactor 110 is the light gas effluent 158 comprising various components, such as water vapor, CO₂, unreacted H₂ and CO, and light hydrocarbons having about one to about six carbon atoms. Light gas effluent 158 can be processed as needed (e.g., water removal) and recycled to the gas plant (not shown). Finally, although not illustrated in FIG. 2, the bottoms of the distillation 155 comprise the heaviest hydrocarbon fraction. This heaviest hydrocarbon fraction comprises semi-solid heavy compounds, such as waxy hydrocarbons typically having greater than about twenty carbon atoms.

Naphtha stream 160, middle distillate stream 162 and the bottoms of the distillation 155 may be fed to an upgrading/refining process (not shown) to form additional commercial valuable products, such as liquid fuels, lubricating oils, and/or waxes. The upgrading/refining process may include one or more hydroconversion units (not shown), such as hydrotreater, hydroisomerization unit and/or hydrocracker, for upgrading streams 160, 162 and the bottoms of the distillation 155. For example, the long-chain waxy hydrocarbons typically present in bottoms of the distillation 155 can be subjected to hydrogenation (olefin saturation) and some chain shortening in the presence of a catalyst and H₂ in a hydrotreater. Alternatively or additionally, bottoms of the distillation 155 can be subjected to thermal degradation/hydrocracking in the presence of a catalyst and H₂ in a hydrocracker, thereby forming converting long-chain hydrocarbon waxes to shorter-chain hydrocarbons boiling in the middle distillate range. Naphtha stream 160, middle distillate stream 162 and the hydroconverted effluent(s) can be further fractioned to form product distillate streams, such as naphtha, kerosene, and diesel. The naphtha, kerosene, and diesel streams are essentially free of sulfur (i.e., less than 10 ppm S, preferably less than 5 ppm S, more preferably less than 1 ppm S) and thus may be used to produce environmentally friendly low-sulfur liquid fuels. The naphtha can be used as a chemical feedstock to make olefins.

The approach used in the present invention to recover a catalyst for reclamation provides several advantages. As mentioned previously, an important economic advantage is that removal of substantially all waxy hydrocarbons from the catalyst reduces the total weight and bulk of the recovered catalyst to be shipped, thereby reducing the cost associated with transporting the recovered catalyst. Additionally, recovering the hydrocarbons in the catalyst slurry and processing them for sale is another economic advantage. Catalyst that is substantially free of hydrocarbons is easier to handle from a health and safety perspective and easier to dispose of in an environmentally sound manner. The present invention does not require an expensive heating process which may also result in the formation of undesirable products. Furthermore, the method of the present invention uses streams within the facility that are readily available and even sometimes considered as waste or undesirable streams under normal operation, i.e., hot gas, naphtha, and nitrogen. Another benefit is that the liquid-solid separation units (such as filters) of the present invention could be used for emergency back up in the case of an upset in the Fischer-Tropsch process. For example, if synthesis gas generation is lost, the slurry comprising the Fischer-Tropsch catalyst and the hydrocarbon liquids could be passed through these liquid-solid separation units so as to separate the liquid wax from the catalyst. This will prevent the mixture of catalyst and wax from cooling and possibly solidifying inside the reactor which could result in an expensive shutdown for manual cleanout of the reactor.

While the preferred embodiments of the invention have been shown and described, modifications thereof can be made by one skilled in the art without departing from the spirit and teachings of the invention. Reactor design criteria, pendant hydrocarbon processing equipment, and the like for any given implementation of the invention will be readily ascertainable to one of skill in the art based upon the disclosure herein. The embodiments described herein are exemplary only, and are not intended to be limiting. Many variations and modifications of the invention disclosed herein are possible and are within the scope of the invention. Use of the term “optionally” with respect to any element of a claim is intended to mean that the subject element is required, or alternatively, is not required. Both alternatives are intended to be within the scope of the claim.

Accordingly, the scope of protection is not limited by the description set out above, but is only limited by the claims which follow, that scope including all equivalents of the subject matter of the claims. Each and every claim is incorporated into the specification as an embodiment of the present invention. Thus the claims are a further description and are an addition to the preferred embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A method for cleaning and recovering a solid catalyst comprising hydrocarbon residue, comprising:
(a) providing a plurality of solvent streams and a plurality of solid-liquid separation units operated in series;
(b) forming a slurry feedstream by adding one of the plurality of solvent streams to a slurry stream comprising a solid catalyst and residual hydrocarbons;
(c) passing the slurry feedstream through a solid-liquid separation unit in the series so as to generate a retentate slurry stream and a liquid stream, wherein the retentate slurry stream comprises the solid catalyst, a portion of the solvent and a lesser content in residual hydrocarbons than that in the slurry feedstream, and wherein the liquid stream comprises a portion of the residual hydrocarbons and the other portion of the solvent;
(d) repeating steps (b) and (c) for each of the downstream solid-liquid separation units in the series, wherein the slurry feedstream for each of the downstream solid-liquid separation units is formed by adding one of the plurality of solvent streams to the retentate slurry stream exiting the solid-liquid separation unit located immediately upstream of said downstream solid-liquid separation unit, said repeating being performed until a desired amount of the residual hydrocarbons is removed from the slurry stream and a catalyst stream exiting the last solid-liquid separation unit in the series comprises primarily the solid catalyst and some solvent;
(e) removing the solvent from the catalyst stream using a stripping gas under a suitable temperature so as to vaporize the solvent and form a cleaned solid catalyst; and
(f) collecting the cleaned solid catalyst.

2. The process of claim 1 wherein the solid catalyst is a Fischer-Tropsch catalyst.

3. The process of claim 2 wherein the Fischer-Tropsch catalyst comprises cobalt.

4. The process of claim 1 wherein the slurry stream in step (b) comprising the solid catalyst is provided from a slurry bubble reactor.

5. The process of claim 1 wherein each of the plurality of solvent streams is a hydrocarbon liquid having a boiling point below the temperature employed in step (e).

6. The process of claim 1 wherein each of the plurality of solvent streams comprises mainly a hydrocarbon material selected from the group consisting of naphtha, diesel, natural gas liquids, and mixtures thereof.

7. The process of claim 1 wherein each of the plurality of solvent streams comprises naphtha.

8. The process of claim 1 wherein each of the plurality of solvent streams is a naphtha stream.

9. The process of claim 8 wherein the naphtha stream is supplied from a distillation column or other available sources within a Fischer-Tropsch process.

10. The process of claim 1 wherein the plurality of solvent streams differ in composition and boiling range.

11. The process of claim 1 wherein each of the plurality of solvent streams is a slipstream from a single solvent feed, which comprises at least a portion of or a fraction of a Fischer-Tropsch liquid product.

12. The process of claim 11 wherein the single solvent feed comprises a naphtha stream derived from a Fischer-Tropsch synthesis.

13. The process of claim 11 wherein the plurality of solvent streams is provided at a temperature of from about 175° C. to about 230° C.

14. The process of claim 1 wherein each of the plurality of solvent streams has a temperature equal to or higher than that of the slurry stream or retentate slurry stream to which it is added.

15. The process of claim 1 wherein the residual hydrocarbons comprise waxy hydrocarbons.

16. The process of claim 15 wherein the waxy hydrocarbons comprise hydrocarbons having equal to or greater than about 20 carbon atoms.

17. The process of claim 1 wherein the plurality of the solid-liquid separation units employs filtration.

18. The process of claim 17 wherein filtration is performed by cross flow filters, rotary filters, cake filters, or any combination thereof.

19. The process of claim 17 wherein filtration comprises permeation of a portion of wax hydrocarbons and solvent across a filter substrate, a catalyst-containing filter cake, or a combination thereof.

20. The process of claim 19 wherein a pressure differential drives the permeation.

21. The process of claim 19 wherein the catalyst-containing filter cake forms on the filter substrate.

22. The process of claim 21 wherein a liquid stream comprising a waxy hydrocarbon and solvent mixture exiting one of the solid-liquid filtration units has a volumetric flow rate of about 3 to about 20% of the total volumetric flow rate of the incoming slurry feedstream to said solid-liquid filtration unit.

23. The process of claim 21 wherein a liquid stream comprising a waxy hydrocarbon and solvent mixture exiting one of the solid-liquid filtration units has a volumetric flow rate of about 8 to about 12% of the total volumetric flow rate of the incoming slurry feedstream to said solid-liquid filtration unit.

24. The process of claim 19 wherein at least one of the liquid streams from one of the solid-liquid filtration units comprises a waxy hydrocarbon and solvent mixture and is fed to a refining section of a Fischer-Tropsch facility.

25. The process of claim 1 wherein the separation steps are repeated until the liquid stream exiting the last solid-liquid separation unit in the series has a boiling point within the range of about 150° C. to about 215° C.

26. The process of claim 1 wherein the plurality of solid-liquid separation units comprises a plurality of filtering steps operated in series.

27. The process of claim 26 wherein the plurality of filtering steps comprises at least about 3 filtering stages in series.

28. The process of claim 26 wherein the plurality of filtering steps comprises at least about 10 filtering stages in series.

29. The process of claim 1 wherein more than 90 percent of residual hydrocarbons are removed from the slurry stream.

30. The process of claim 29 wherein more than 95 percent of residual hydrocarbons are removed from the slurry stream.

31. The process of claim 1 wherein the stripping gas comprises one or more gases selected from the group consisting of hydrogen, methane, nitrogen, natural gas, and super-heated steam.

32. The process of claim 31 wherein the stripping gas comprises an off-gas supplied from one or more sources selected from the group consisting of a natural gas separation unit, a refining process, a hydroprocessing unit, and a Fischer-Tropsch reactor.

33. The process of claim 31 wherein the stripping gas comprises more than 50% by volume of methane.

34. The process of claim 1 wherein the stripping gas is supplied at a temperature from 200° C. to 290° C.

35. The process of claim 1 wherein each of the solvent streams is a naphtha stream, and the stripping gas has an end boiling point which is lower than that of said naphtha stream.

36. The process of claim 1 further comprising degassing the stripped catalyst stream to remove any remaining residual hydrocarbons or naphtha.

37. The process of claim 36 further comprising feeding the stripping gas or remaining residual hydrocarbons or naphtha to a refining unit for further processing.

38. The process of claim 1 wherein at least one of the solid-liquid separation units comprises a liquid-liquid extraction zone having one or more filters disposed therein.

39. A system for cleaning a solid catalyst comprising hydrocarbon residue to obtain a cleaned catalyst suitable for reclamation, comprising:
(a) means for diluting a slurry comprising a solid catalyst and residual hydrocarbons with a solvent;
(b) a plurality of solid-liquid separation units in series configured for receiving a diluted slurry and separating substantially all of the residual hydrocarbons from the solid catalyst so that each of the plurality of solid-liquid separation units forms a liquid stream and a retentate slurry, wherein the diluted slurry for any of the downstream solid-liquid separation units comprises the retentate slurry from the solid-liquid separation unit immediately upstream of said downstream solid-liquid separation unit; and
(c) a stripper connected to the last solid-liquid separation unit in the series for receiving the retentate slurry exiting from the last solid-liquid separation unit and configured for passing a stripping gas so as to remove any residual hydrocarbons and solvent from the solid catalyst present in the retentate slurry exiting from the last solid-liquid separation unit and form a cleaned catalyst.

40. The system of claim 39 wherein the slurry is provided from a slurry bubble Fischer-Tropsch reactor.

41. The system of claim 39 wherein the solid catalyst is a particulate Fischer-Tropsch catalyst.

42. The system of claim 39 wherein the solid catalyst comprises cobalt and optionally one or more metal promoters.

43. The system of claim 39 further comprising a plurality of Fischer-Tropsch reactor vessels manifolded to provide the slurry.

44. The system of claim 39 wherein the solid-liquid separation units in series comprise filters.

45. The system of claim 44 wherein the filters are cross flow filters, rotary filters, cake filters, or any combination thereof.

46. The system of claim 39 further comprising a degasser connected to the stripper and configured for receiving and degassing any vaporized residual hydrocarbons, solvent, or stripping gas remaining with the solid catalyst.

47. The system of claim 39 wherein at least one of the solid-liquid separation units comprises a liquid-liquid extraction zone having one or more filters disposed therein.

48. An integrated process for producing hydrocarbons, comprising:
(a) contacting a synthesis solid catalyst with a feed stream comprising carbon monoxide and hydrogen in a reaction zone within a reactor to produce hydrocarbon products until all or a portion of the solid catalyst needs to be replaced;
(b) removing all or a portion of the solid catalyst from the reactor via a slurry comprising said solid catalyst and hydrocarbons;
(c) adding a solvent to the slurry to form a diluted slurry;
(d) separating a portion of the hydrocarbons and the solvent from the diluted slurry;
(e) repeating steps c and d until a desired amount of the hydrocarbons have been removed such as to generate a catalyst stream comprising primarily the solid catalyst and some solvent;
(f) removing the solvent from the catalyst stream using a stripping gas to form a cleaned solid catalyst;
(g) recovering the cleaned solid catalyst; and
(h) replacing the removed catalyst with fresh catalyst.

49. The process of claim 48 wherein the reactor is a slurry bed reactor.

50. The process of claim 48 wherein the catalyst slurry is removed from a catalyst recirculation loop connected to the slurry bed reactor.

51. The process of claim 48 wherein the solid catalyst comprises a Fischer-Tropsch catalyst.

52. The system of claim 48 wherein the solid catalyst comprises cobalt and optionally one or more metal promoters.

53. The process of claim 48 wherein the hydrocarbons in the slurry comprise residual waxy hydrocarbons.

54. A method for unloading the content of a slurry bubble reactor comprising a solid catalyst and a waxy hydrocarbon liquid with minimal solidification of the waxy hydrocarbon liquid to recover the solid catalyst, the method comprising the steps of:
(a) passing a feed gas comprising hydrogen and carbon monoxide as reactant gases through a slurry being maintained in the reactor under conversion promoting conditions which include a reaction temperature between about 160° C. and 300° C. to convert at least a portion of said reactant gases to hydrocarbon products, wherein the slurry comprises a solid catalyst and a molten waxy hydrocarbon liquid;
(b) substituting one or both of the reactant gases by an unreactive gas or removing one of the reactant gases so as to stop the hydrocarbon synthesis reaction;
(c) periodically or continuously adding a lighter diluting hydrocarbon liquid to the slurry in the reactor so as to gradually reduce the content of the molten waxy hydrocarbon liquid in the slurry;
(d) optionally, cooling the slurry within the slurry bubble column reactor from the reaction temperature to a lower temperature;
(e) withdrawing a slurry stream from the reactor and passing the slurry stream through an external slurry circulation loop comprising a solid-liquid separation unit to form a solid-enriched slurry stream and a hydrocarbon product stream which exits the external slurry circulation loop;
(f) recycling the majority of or all of the solid-enriched slurry stream to said slurry bubble column reactor,
(g) performing steps (c), (e), (f) and optionally (d) until the slurry contained in the reactor has a lower waxy hydrocarbon content and has an acceptable temperature without causing solidification of the slurry within the reactor; and
(h) withdrawing a part of or all of the wax-reduced slurry from the reactor to feed a recovery system to recover the solid catalyst therein.

55. The method of claim 54 wherein the acceptable temperature ranges from ambient temperature to about 160° C.

56. The method of claim 54 wherein the acceptable temperature ranges from ambient temperature to about 120° C.

57. The method of claim 54 wherein the solid-liquid separation unit comprises one or more filters.

58. The method of claim 54 wherein the solid-liquid separation unit further comprises a degasser to remove entrapped gas from the slurry stream.

59. The method of claim 54 wherein the reaction temperature ranges between about 190° C. and 260° C.

60. The method of claim 54 wherein the lighter diluting hydrocarbon liquid comprises a hydrocarbon mixture within the diesel, kerosene, or lubricating oil boiling range.

61. The method of claim 54 wherein the lighter diluting hydrocarbon liquid comprises a hydrocarbon mixture within the naphtha boiling range.

62. The method of claim 54 wherein step (b) comprises substituting one or both of the reactant gases by an unreactive gas.

63. The method of claim 54 wherein step (d) is performed continuously or intermittently while step (c) is performed.

64. The method of claim 54 wherein step (d) is performed continuously or intermittently.

65. The method of claim 54 while step (d) is performed while step (c) is performed.

66. The method of claim 54 wherein step (d) is performed before step (b).
